SEQUENCE LISTING (5 sequences)

SEQ ID NO: 1 - DNA, 25 bp, Unknown
  Mammal
taagcttggcacggctgtccaagga

SEQ ID NO: 2 - DNA, 26 bp, Unknown
  Mammal
acagaattcgccccggcctggtacac

SEQ ID NO: 3 - DNA, 19 bp, Unknown
  Mammal
gtctccaactgctgacctc

SEQ ID NO: 4 - DNA, 19 bp, Unknown
  Mammal
ctgccttttcaagcctcag

SEQ ID NO: 5 - DNA, 18 bp, Unknown
  Mammal
tacaggccacaaatgtga

(19) United States
(12) Patent Application Publication
(10) Pub. No.: US 2012/0231457 A1
(43) Pub. Date: Sep. 13, 2012

(54) COMPOSITIONS AND METHODS FOR ASSESSING A GENETIC RISK OF DEVELOPING LATE-ONSET ALZHEIMER'S DISEASE (LOAD)

(76) Inventors: Nikolaos Tezapsidis, West Orange, NJ (US); Steven J. Greco, Carlstadt, NJ (US)

(21) Appl. No.: 13/415,940

(22) Filed: Mar. 9, 2012

Related U.S. Application Data
(60) Provisional application No. 61/451,439, filed on Mar. 10, 2011.

(51) Int. Cl. C12Q 1/68

(52) U.S. Cl. 435/6.11

(57) ABSTRACT

The described invention provides compositions and methods for assessing a genetic risk of developing late-onset Alzheimer's disease (LOAD) in a subject by analyzing haplotypes of human Apolipoprotein E (APOE) and Translocase of Outer Mitochondrial Membrane 40 homolog (TOMM40) genes using a PCR- and restriction digest-based approach.

### CROSS REFERENCE TO RELATED APPLICATIONS

This Application claims the benefit of priority to U.S. Provisional Application No. 61/451,439 (filed Mar. 10, 2011) entitled “Isotyping the Human TOMM40 Variable-Length Polymorphism by Gene Amplification and Restriction Digest,” the entire contents of which are incorporated by reference herein.

### STATEMENT OF GOVERNMENT FUNDING

The described invention was made with government support under Grant No. SBIR-1R43AG029670 from the National Institute on Aging. The government has certain rights in the invention.

### FIELD OF THE INVENTION

The described invention relates to methods for assessing a genetic risk of developing late-onset Alzheimer's disease (LOAD).

### BACKGROUND OF THE INVENTION

Alzheimer's Disease

Alzheimer's disease (also called “AD”, “senile dementia of the Alzheimer Type (SDAT)” or “Alzheimer's”) is a neurodegenerative disorder of the central nervous system (“CNS”). AD is usually diagnosed clinically from the patient history, collateral history from relatives, and clinical observations, based on the presence of characteristic neurological and neuropsychological features.

The pathology of AD includes, but is not limited to, (1) missense mutations in APP, PS1 and PS2 genes; (2) altered proteolysis of Aβ42; (3) progressive accumulation and aggregation of Aβ42 in brain interstitial fluid; (4) deposition of aggregated Aβ42 as diffuse plaques (in association with proteoglycans and other amyloid-promoting substrates); (5) aggregation of Aβ40 onto diffuse Aβ42 plaques and accrual of certain plaque-associated proteins (such as, for example, complement clq etc); (6) an inflammatory response including (a) microglial activation and cytokine release, (b) astrocytosis and acute phase protein release; (7) progressive neuritic injury within amlyoid plaques and elsewhere in the neuropil; (8) disruption of neuronal metabolic and ionic homeostasis; (9) oxidative injury; (10) altered kinase/phosphatase activities leading to hyperphosphorlyated tau which leads to paired helical filament (PHF) formation; (11) widespread neuronal/neuritic dysfunction and death in the hippocampus and cerebral cortex with progressive neurotransmitter deficits; and (12) dementia. The ultimate effects that may further present in the affected cortical regions are neuritic dystrophy, synaptic loss, shrinkage of neuronal perikarya, and selective neuronal loss.

AD is further characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Both amyloid plaques (“AP”) and neurofibrillary tangles (“NFT”) are clearly visible by microscopy in brains of those afflicted with AD.

Amyloid β (Aβ)

Amyloid β is derived from its large precursor protein (APP) by sequential proteolytic cleavages. APP comprises a heterogeneous group of ubiquitously expressed polypeptides. This heterogeneity arises both from alternative splicing (yielding 3 major isoforms of 695, 751 and 770 residues) as well as by a variety of posttranslational modifications, including the addition of N- and O-linked sugars, sulfation, and phosphorylation. The APP splice forms containing 751 or 770 amino acids are widely expressed in normeuronal cells throughout the body and also occur in neurons. However, neurons express even higher levels of the 695 residue isoform, which occurs at very low abundance in normeuronal cells. The difference between the 751/770-residue and 695-residue forms is the presence in the 751/770-residue isoform of an exon that codes for a 56-amino acid motif that is homologous to the Kunitz-type of serine protease inhibitors (KPI), indicating one potential function of these longer APP isoforms. The KPI-containing isoforms of APP found in human platelets serve as inhibitors of factor Xia, which is a serine protease in the coagulation cascade. APP is highly conserved in evolution and is expressed in all mammals examined for it; a partial homolog of APP has been found in Drosophilia (APPL). APP is a member of a larger gene family, the amyloid precursor-like proteins (APLPs) which have substantial homology, both within the large ectodomain and the cytoplasmic tail, but are divergent in the Aβ region.

APP is a single transmembrane polypeptide that is co-translationally translocated into the endoplasmic recticulum via its signal peptide and then post-translationally modified through the secretory pathway. Its acquisition of N- and O-linked sugars occurs rapidly after biosynthesis, and its half-life is relatively brief (45 to 60 minutes). Both during and after the trafficking of APP through the secretory pathway, APP can undergo a variety of proteolytic cleavages to release secreted derivatives into vesicle lumens and the extracellular space. The first proteolytic cleavage identified, that made by an activity designated α-secretase, occurs 12 amino acids NH₂-terminal to the single transmembrane domain of APP. This processing results in the release of the large soluble ectodomain fragment (β-APPₛ) into the lumen/extracellular space and retention of an 83-residue COOH-terminal fragment (CTF) in the membrane. Alternatively, some APP molecules not subjected to α-secretase cleavage can be cleaved by an activity designated β-secretase, which principally cuts 16 residues NH₂-terminal to the α-cleavage site, generating a slightly smaller ectodomain derivative (β-APPs) and retaining a 99-residue CFT (C99) in the membrane that begins at residue 1 of the Aβ region. The C99 fragment is consequently cleaved in the middle of the transmembrane domain as a result of γ-secretase. Accordingly, Aβ production is a normal metabolic event; precisely where during its complex intracellular trafficking APP can undergo the α-, β- and γ-secretase remains unknown.

In polarized epithelial cells, such as Madin-Darby Canine Kidney (MDCK) cells, APP is principally targeted to the basolateral membrane, where it can undergo α-secretase cleavage to release α-APPₛ basolaterally, although a small fraction is targeted and processed apically. In neurons, which are one of the cells that express the highest levels of APP in the body (particularly APP695), APP can be anterogradely transported in the fast component of axonal transport. APP is present in vesicles in axonal terminals, although not specifically in synaptic vesicles. Cell biological studies demonstrate that APP in the axonal terminals can be retrogradely transported up the axon to the cell body, and some molecules are then fully translocated to the somatodendritic surface. During its retrograde axonal trafficking, some APP molecules can be recycled to the axolemmal surface. Although it has been assumed that APP axonal terminals might be a principal site for the generation of Aβ, this has not been definitely determined, and APP that recycles in endosomes at various neuronal subsites may be capable of undergoing the sequential β- and γ-secretase cleavages to release the peptide. Although APP is particularly abundantly expressed in neurons and they have been shown to secrete substantial amounts of Aβ peptides, other brain cells also express APP and release variable amounts of Aβ, including astrocytes, microglia, and endothelial and smooth muscle cells, and these could all contribute to the secreted pool of Aβ that eventually leads to extracellular deposition. Moreover, the fact that virtually all peripheral cells also express APP and generate Aβ and that Aβ is present in plasma raises the possibility that circulating Aβ could cross the blood-brain barrier and contribute to cerebral Aβ accumulation.

A number of functions have been ascribed to APP holoproteins and/or their major secreted derivative (α-APPₛ) based on cell culture studies. Soluble α-APPs was shown to be capable of acting as an autocrine factor and a neuroprotective and perhaps neurotrophic factor. In vitro studies indicate that the 751- and 770-residue isoforms (encoding a KPI motif) inhibit serine proteases such as trypsin and chymotrypsin. The secreted APP isoforms can confer cell-cell and cell-substrate adhesive properties in culture. All of these imputed functions have not yet been confirmed in vivo.

### Lipid Rafts

It generally is believed that brain lipids are intricately involved in Aβ-related pathogenic pathways. The Aβ peptide is the major proteinaceous component of the amyloid plaques found in the brains of AD patients and is regarded by many as the culprit of the disorder. The amount of extracellular Aβ accrued is critical for the pathobiology of AD and depends on the antagonizing rates of its production/secretion and its clearance. Studies have shown that neurons depend on the interaction between Presenilin 1 (“PS1”) and Cytoplasmic-Linker Protein 170 (“CLIP-170”) to both generate Aβ and to take it up through the lipoprotein receptor related protein (“LRP”) pathway. Further to this requirement, formation of Aβ depends on the assembly of key proteins in lipid rafts (“LRs”). Within the LRs it is believed that APP is cleaved first by the β-secretase (BACE) to generate the C-terminal intermediate fragment of APP (CAPPβ), which remains embedded in the membrane. CAPPβ subsequently is cleaved at a site residing within the lipid bilayer by γ-secretase, a high molecular weight multi-protein complex containing presenilin, (PS1/PS2), nicastrin, PEN-2, and APH-1 or fragments thereof. Aβ finally is released outside the cell where it may: (i) start accumulating following oligomerization and exerting toxicity to neurons, or (ii) be removed either by mechanisms of endocytosis (involving apolipoprotein-E (apoE) and LRP or Scavenger Receptors) or by degradation by extracellular proteases including insulin-degrading enzyme (IDE) and neprilysin.

### Principal Underyling Cause of Alzheimer's Disease Remains Unknown

The principal underlying cause of AD remains unknown. Disagreements persist as to whether Aβ peptide-rich plaques or neurofibrillary tangles (NFTs) are the principal neurodegenerative element and whether they are etiologically related. There is a high degree of disparity among research efforts to address whether there are earlier biochemical events that ultimately lead to the characteristic pathology. It generally is believed that soluble Aβ oligomers, prior to plaque buildup, exert neurotoxic effects leading to neurodegeneration, synaptic loss, and dementia. Further, increased Aβ levels may result from abnormal lipid accumulation, thereby producing altered membrane fluidity and lipid raft composition. However, for sporadic AD, representing the overwhelming majority of AD cases, there still is no convincing evidence for a particular cause that triggers the Aβ cascade.

Clinical diagnosis of late onset Alzheimer's disease (LOAD) principally relies on imaging- and neuropsychological-based screening tools, which are adept at identifying stages of the disease already presenting significant pathophysiological advancement (Schroeter et al., Neuroimage, 47: 1196-206, 2009; Waldemar et al., Eur J Neurol, 14: e1-262007, 2007)

The presence of disease-related biomarkers in the cerebrospinal fluid (CSF), such as β-amyloid (Aβ(1-42)), total and/or phosphorylated tau (phospho-tau181), has been shown to substantially assist in the accurate identification of AD cases, supporting clinical diagnosis and potentially allowing earlier diagnoses (De Meyer et al., Arch Neurol; 67: 949-56, 2010). However, consideration of CSF collection via lumbar puncture can be disquieting to subjects, and these biomarkers cannot be used for early AD clinical diagnosis as a stand-alone method. Work from a number of laboratories focused on the identification of surrogate biomarkers in CSF or blood plasma for early diagnosis of AD has met some success (Rupsingh et al., Neurobiol Aging, 32: 802-810, 2009)

It is becoming apparent that CSF-Aβ(1-42) declines for years prior to the development of dementia, with the period of decline occurring progressively earlier depending on genotype-related risk, while CSF-tau declines in relationship to the impairment of memory and the development of dementia. However, larger number of patients for longer follow-up periods are required to verify the usefulness of these markers in accurately predicting the progression of subjects from mild cognitively impairment (MCI) to dementia.

A small number of familial cases of AD (FAD) are characterized by an early-onset form of the disease that is inherited through mutations in three genes (PS1, PS2 and APP) in a fully-penetrate dominant fashion. It is well accepted that among the LOAD cases other genetic factors may confer susceptibility to the disease and/or contribute to earlier onset of the disease. Ideally, a hallmark AD genotype would be able to predict risk to convert to AD in pre-symptomatic patients, thereby allowing a preventative approach. To date, no genetic risk factors carry the same prognostic power for LOAD as observed in familial AD. Genome-wide association studies have shown a limited number of genes correlating with the disease (Waring and Rosenberg, Arch Neurol, 2008; 65: 329-334).

### Genetic Risk Factors for Late-Onset Alzheimer's Disease (LOAD)

Early diagnosis and/or determination of genetic risk factors for late-onset Alzheimer's disease (LOAD) are predicted to become pivotal, once effective disease modification treatments for the disorder will be available. Currently, there are no stand-alone, reliable/definite (epigenetic or genetic) biomarkers, which are predictive for LOAD. Confidence in early diagnoses relies on the compilation of data including CSF and plasma biomarkers, brain imaging, and cognition-based tools. Genetic tests afford another diagnostic measure to assess LOAD vulnerability, but only a few genes have been linked to LOAD.

The most well known of these genetic risk factors is inheritance of the ε4 allele of the apolipoprotein E (APOE) gene of which homozygotes are fifteen times more likely to develop AD than non-carriers (Ashford et al., Lancet, 2006; 368: 387-403). Apolipoprotein E (APOE), differs by a single nucleotide at amino acid positions 112 and 158 to generate three isoforms, ε2, ε3 and ε4, with the ε4 isotype showing prevalence in AD. However, carriers of the ε3 allele, which are at greater frequency in the global population (Eisenberg et al., Am J Phys Anthropol; 143: 100-11, 2010) are still susceptible to developing LOAD. Of considerable interest, the carriers of the ε2 allele have a progressively decreased Alzheimer risk.

Recent studies also have shown that the Translocase of Outer Mitochondrial membrane 40 homolog (TOMM40) gene contains a polymorphic poly-T variant that could subdivide ε3 carriers into two risk groups for AD (Roses et al., Pharmacogenomics J, 10: 375-84, 2010). Presumably, one of the groups has a form which carries a risk more similar to the ε4 allele, while the other may be associated with a risk more similar to that of the ε2 allele. This novel conceptualization could enhance our understanding about the genetics associated with LOAD. The framework for this study of TOMM40 was based on the identification and association of a region of linkage disequilibrium involving three genes, APOE, TOMM40 and APOC1, with LOAD (Martin et al., Am J Hum Genet, 67: 383-394, 2000; Takei et al., Genomics, 93: 441-448, 2009; Yu et al., Genomics, 2007; 89: 655-65)

Development of a TOMM40 assay to genotype polymorphic variants can be used to stratify patients enrolled in clinical trials for AD. ε4/ε4 genotypes are generally considered the most at-risk groups for LOAD. Now, ε3/ε4 and potentially ε3/ε3 (and even ε2/ε3) genotypes can also be considered at increased risk for early disease onset depending on the presence of the TOMM40 long/very long variant. To date, TOMM40 association studies have not been performed in a large enough sample size or a number of ethnically diverse populations to know whether the AD link holds true globally. However, recently, it was announced that Takeda Pharmaceuticals will use a TOMM40 assay developed by Zinfandel Pharmaceuticals as part of a clinical trial investigating the utility of Takeda's type 2 diabetes drug Actos (pioglitazone) in AD. The assay will stratify treatment groups for at-risk older adults with normal cognition based on APOE/TOMM40 haplotype. The need for stratification of patients based on their APOE genotype has been suggested from previous clinical trials of rosiglitazone (Gold et al., Dement Geriatr Cogn Disord; 30: 131-146, 2010; Risner et al., Pharmacogenomics J, 2006; 6: 246-254).

Current methods to isotype the poly-T region rely on long PCR, subcloning, and sequencing to distinguish among the allelic variants. While such methods are extremely accurate as well as quantitative in determining the number of T residues in the poly-T region the process can be cumbersome, time consuming, and expensive to employ in routine laboratories, especially when utilized for analysis of a large number of patient samples.

The described invention provides a quick and simple alternative method to isotype the human APOE and TOMM40 variable-length polymorphisms using a PCR- and restriction digest-based approach, which enables rapid, qualitative genotyping of APOE/TOMM40 variants with one sample in the same workflow. Although this method does not quantify the exact number of T residues in each polymorphic variant, it can identify individual isotypes as short, long or very long, as well as distinguish homo- and heterozygousity. This qualitative distinction among two (short, long) or three (short, long, very long) variants has potential to be a valuable and easily implemented tool for LOAD risk assessment and clinical trial design.

### SUMMARY OF THE INVENTION

According to one aspect, the described invention provides a method for assessing a genetic risk of developing late-onset Alzheimer's disease (LOAD) in a subject, the method comprising: (a) isolating a genomic deoxyribonucleic acid (gDNA) from the subject; (b) amplifying a first genomic region of the genomic deoxyribonucleic acid (gDNA) from step (a) using a first forward primer (5′-TAA GCT TGG CAC GGC TGT CCA AGG A-3′; SEQ ID NO: 1) and a first reverse primer (5′-ACA GAA TTC GCC CCG GCC TGG TAC AC-3′; SEQ ID NO: 2), wherein the first genomic region comprises genomic deoxyribonucleic acid (gDNA) encoding amino acid positions 112 and 158 of Apolipoprotein E (APOE), wherein Apolipoprotein E (APOE) isoforms ε2, ε3, and ε4 contain two single nucleotide polymorphisms (SNPs) at the amino acid positions 112 and 158 of Apolipoprotein E (APOE), wherein the first forward primer and the first reverse primer flank the two single nucleotide polymorphisms (SNPs), and wherein the amplification produces a first amplified deoxyribonucleic acid (DNA) with a length of 244 base pair; (c) amplifying a second genomic region of the genomic deoxyribonucleic acid (gDNA) from step (a) using a second forward primer (5′-GTC TCC AAC TGC TGA CCT C-3′; SEQ ID NO: 3) and a second reverse primer (5′-CTG CCT TTT CAA GCC TCA G-3′; SEQ ID NO: 4), wherein the second genomic region comprises intron 6 of Translocase of Outer Mitochondrial Membrane 40 homolog gene (TOMM40) containing a polymorphic region of poly-thymidine (poly-T), wherein the second forward primer and the second reverse primer flank the polymorphic region of the Translocase of Outer Mitochondrial Membrane 40 homolog gene (TOMM40), and wherein the amplification produces a second amplified deoxyribonucleic acid (DNA) with a length of from about 360 base pair to about 390 base pair; (d) digesting the first amplified deoxyribonucleic acid (DNA) from step (b) with restriction enzyme HhaI, wherein the restriction enzyme HhaI differentially cleaves the two single nucleotide polymorphisms (SNPs) located at amino acid positions 112 and 158 of Apolipoprotein E (APOE) thereby produces a first Restriction Fragment Length Polymorphism (RFLP) comprising: (i) a 72 base pair fragment, (ii) an 81 base pair fragment or (iii) absence of either the 72 base pair fragment or the 81 base pair fragment; (e) digesting the second amplified deoxyribonucleic acid (DNA) from step (c) with restriction enzyme SmaI, wherein the restriction enzyme SmaI produces a second Restriction Fragment Length Polymorphism (RFLP) comprising (i) a constant length restriction fragment of about 230 base pair independent of the poly-thymidine (Poly-T) region, and (ii) a variable-length restriction fragment with a nucleotide length of from about 130 to about 160 base pair; (f) analyzing haplotypes of the Apolipoprotein E gene (APOE) in the subject based on the first restriction fragment length polymorphism (RFLP) produced by step (d), wherein presence of the 72 base pair fragment indicates that the subject has an ε4 allele for the Apolipoprotein E gene (APOE), wherein presence the 81 base pair fragment indicates that the subject has an ε2 allele for the Apolipoprotein E gene (APOE), and wherein absence of either the 72 base pair fragment or the 81 base pair fragment indicates that the subject has an ε3 allele for the Apolipoprotein E gene (APOE); (g) analyzing haplotypes of Translocase of Outer Mitochondrial Membrane 40 homolog gene (TOMM40) in the subject based on the second restriction fragment length polymorphism (RFLP) produced by step (e), wherein presence of the variable-length restriction fragment of between 15 and 19 thymidine residues indicates that the subject has a short poly-T allele for the Translocase of Outer Mitochondrial Membrane 40 homolog gene (TOMM40), wherein presence of the variable-length restriction fragment of between 20 and 29 thymidine residues indicates that the subject has a long poly-T allele for the Translocase of Outer Mitochondrial Membrane 40 homolog gene (TOMM40), wherein presence of the variable-length restriction fragment of between 30 and 39 thymidine residues indicates that the subject has a very long poly-T allele for the Translocase of Outer Mitochondrial Membrane 40 homolog gene (TOMM40); and (h) determining the subject's genetic risk for late-onset Alzheimer's disease (LOAD) based upon the haplotype analysis of the Apolipoprotein E gene (APOE) and the Translocase of Outer Mitochondrial Membrane 40 homolog gene (TOMM40) obtained from step (f) and step (g), wherein the subject is diagnosed as being in a most at-risk group for late-onset Alzheimer's disease (LOAD) if homozygous ε4 alleles (ε4/ε4) for the Apolipoprotein E gene (APOE) are present in the subject, wherein the subject is diagnosed as being in an increased risk group for late-onset Alzheimer's disease (LOAD) if heterozygous ε3 and ε4 alleles (ε3/ε4) for the Apolipoprotein E gene (APOE), homozygous ε3 alleles (ε3/ε3) for the Apolipoprotein E gene (APOE), or heterozygous ε2 and ε3 alleles (ε2/ε3) for the Apolipoprotein E gene (APOE) are present together with either the long poly-T allele or the very long poly-T allele for the Translocase of Outer Mitochondrial Membrane 40 homolog gene (TOMM40) in the subject.

According to one embodiment of the method, the genomic deoxyribonucleic acid (gDNA) is isolated from cerebrospinal fluid (CSF) of the subject. According to another embodiment, the genomic deoxyribonucleic acid (gDNA) is isolated from peripheral blood of the subject. According to another embodiment, amplification steps (b) and (c) are carried out by a polymerase chain reaction (PCR). According to another embodiment, amplification steps (b) and (c) are carried out in a single polymerase chain reaction (PCR). According to another embodiment, digesting steps (d) and (e) are carried out in a single restriction reaction. According to another embodiment, digestion step (d) is carried out using an isoschizomer of the restriction enzyme HhaI. According to another embodiment, digestion step (e) is carried out using an isoschizomer of the restriction enzyme SmaI. According to another embodiment, in step (h) the subject in the increased risk group for late-onset Alzheimer's disease (LOAD) has heterozygous ε3 and ε4 alleles (ε3/ε4) for the Apolipoprotein E gene (APOE) gene together with either the long poly-T allele or the very long poly-T allele for the Translocase of Outer Mitochondrial Membrane 40 homolog gene (TOMM40). According to another embodiment, in step (h) the subject in the increased risk group for late-onset Alzheimer's disease (LOAD) contains homozygous ε3 alleles (ε3/ε3) for the Apolipoprotein E gene (APOE) together with either the long poly-T allele or the very long poly-T allele for the Translocase of Outer Mitochondrial Membrane 40 homolog gene (TOMM40). According to another embodiment, in step (h) the subject in the increased risk group for late-onset Alzheimer's disease (LOAD) contains heterozygous ε2 and ε3 alleles (ε2/ε3) for the Apolipoprotein E gene (APOE) together with either the long poly-T allele or the very long poly-T allele for the Translocase of Outer Mitochondrial Membrane 40 homolog gene (TOMM40).

### BRIEF DESCRIPTION OF THE FIGURES

FIG. 1 shows a polymerase chain reaction (PCR) and restriction digest of APOE polymorphic region. (A) Genomic DNA extracted from human hippocampal tissue was used as a template for PCR with APOE-specific primers spanning the APOE single nucleotide polymorphism (SNP) region. The resulting amplicon is seen at 244 bp. (B) The PCR product was subsequently digested with HhaI and run on a 4% agarose gel to generate a pattern of restriction fragments corresponding to SNPs at amino acid positions 112 and 158 of APOE. These Restriction Fragment Length Polymorphisms (RFLPs) were then used to determine the APOE genotype. Illustrated samples serve as a representative sample population of the 22 subjects.

FIG. 2 shows a cartoon depicting the process used to generate variable size fragments containing the TOMM40 poly thymidine (poly-T) region. Genomic DNA extracted from human hippocampal tissue was used as a template for PCR to amplify the intronic region (intron 6) between exons 6 and 7 of the TOMM40 gene, which spans a poly-T region of variable length. The resulting PCR product of between 360-390 bp, depending on poly-T length, was digested with SmaI to generate a constant restriction fragment (˜230 bp) independent of poly-T length and a variable fragment containing the poly-T site (˜130-160 bp). The size of the variable fragment can then be used to determine whether each allele contains the short (<20 T residues) and/or the long (>20 T residues) isoforms.

FIG. 3 shows PCR and restriction digest of TOMM40 intronic poly-T region. (A) Amplification of genomic DNA (gDNA) using primers spanning the TOMM40 poly-T region (FIG. 2) generated a PCR product of between 360-390 bp. (B) To determine the TOMM40 genotype, the PCR product was subsequently digested with SmaI and run on a 4% agarose gel to generate a constant fragment of 230 bp and a variable fragment of between 130-160 bp. The allelic variation in the size of the poly-T-containing fragment(s) was used to determine homo- and heterozygosity. Arrows identify a heterzygote whose variants differ by few base pairs. L represents long variant and S represents short variant.

FIG. 4 shows confirmatory sequencing and alignment of a TOMM40 homozygous variant. To confirm the identity of the TOMM40 PCR product, a homozygous TOMM40 variant (A92-218) was sequenced to identify the constant region and to determine T residue length in the variable region. A homozygous subject was chosen to preclude sequencing errors from allelic variability at the poly-T site seen in heterozygotes.

### DETAILED DESCRIPTION OF THE INVENTION

### Glossary

The term “allele” refers to an alternative form of a gene.

The term “amplify” as used herein refers to exponentially making copies of a fragment or sequence of DNA.

The term “amplicon” as used herein refers to double stranded DNA fragments produced by PCR amplification of a segment of DNA.

The terms “amyloid peptide” “amyloid β peptide” and “Aβ” are used interchangeably herein to refer to the family of peptides generated through proteolytic processing of amyloid precursor protein (APP).

The term “anneal” as used herein refers to joining of single strands of DNA via pairing of complementary bases. In PCR, primers anneal to complementary target DNA sequences during cooling of the DNA (after DNA is made single stranded by heating).

The term “dementia” as used herein refers to a decline or a progressive decline in cognitive function due to damage or disease in the brain beyond what might be expected from normal aging. The term “cognitive function” refers to the intellectual processes resulting in an understanding, perception, or awareness of one's ideas as well as the ability to perform mental tasks, such as thinking, learning, judging, remembering, computing, controlling motor functions, and the like.

The term “fragment” as used herein refers to an isolated portion of a nucleic acid.

The term “gene” as used herein refers to a locatable segment of a genomic sequence corresponding to a unit of inheritance, which is associated with regulatory regions, transcribed regions that code for a protein or RNA product, and other functional sequence regions.

The terms “gene expression” and “expression” are used interchangeably herein to refer to the process by which inheritable information from a gene, such as a DNA sequence, is made into a functional gene product, such as protein or RNA.

The term “genomic DNA” or “gDNA” as used herein refers to DNA that is derived from a genome. The term “genome” means the whole hereditary information of a species, which is encoded in DNA. The term “genomic DNA” or “gDNA” as used herein encompasses genomic RNA or nucleolar RNA, non-spliced RNA or partially spliced RNA. RNA samples may be transcribed into DNA samples by processes such as reverse transcription for the purpose of the described invention.

The term “haplotype” as used herein refers to a combination of alleles at loci that are found on a single chromosome or DNA molecule.

The term “hybridization” refers to the binding of two single stranded nucleic acid molecules to each other through base pairing. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable.

The term “isoschizomer” as used herein refers to a restriction endonuclease enzyme that recognizes and binds to the same recognition sequence as another restriction endonuclease, but is isolated from different microbial sources.

The term “isotype” as used herein refers to genetic variations or differences

An “isolated molecule” as used herein refers to a molecule that is substantially pure or essentially free of other substances with which it is ordinarily found in nature or in vivo systems to an extent practical and appropriate for its intended use. As used herein, the term “substantially pure” refers to purity of at least 75%, at least 80%, at least 85%, at least 90%, at least 95% or at least 99% pure as determined by an analytical protocol. The term “substantially free” or “essentially free” are used herein to refer to considerably or significantly free of, or more than about 75%, 80%, 85%, 90%, 95%, or more than about 99% free of.

The term “lipid rafts” as used herein refers to membrane microdomains enriched in cholesterol, glycosphingolipids and glucosylphosphatidyl-inositol-(GPI)-tagged proteins implicated in signal transduction, protein trafficking and proteolysis.

The term “nucleic acid” as used herein refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” as used herein refers to a chemical compound that consists of a heterocyclic base, a sugar, and one or more phosphate groups. In the most common nucleotides the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. The purines include adenine (A), and guanine (G); the pyrimidines include cytosine (C), thymine (T), and uracil (U).

The term “polymerase chain reaction” or “PCR” refers to a technique to replicate a desired segment of a nucleic acid. PCR starts with primers that flank the desired target fragment of a nucleic acid. The nucleic acid strands are first separated with heat, and then cooled allowing the primers bind to their target sites. Polymerase then makes each single strand into a double strand, starting from the primer. This cycle is repeated multiple times.

The term “polynucleotide” refers to a deoxyribopolynucleotide, ribopolynucleotide, or an analog thereof that has the essential nature of a natural deoxyribopolynucleotide or ribonucleotide in that it hybridizes, under stringent hybridization conditions, to substantially the same nucleotide sequence as naturally occurring nucleotides and/or allow translation into the same amino acid(s) as the naturally occurring nucleotide(s). A polynucleotide may be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes are known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The term “primer” refers to a nucleic acid which, when hybridized to a strand of DNA, is capable of initiating the synthesis of an extension product in the presence of a suitable polymerization agent. The primer is sufficiently long to uniquely hybridize to a specific region of the DNA strand. A primer also may be used on RNA, for example, to synthesize the first strand of cDNA.

The term “polymorphism” refers to the presence of more than one allele at a locus. Polymorphism is also used as a measure of the proportion of loci in a population that are genetically variable or polymorphic (P).

The term “DNA polymorphism” is used to describe a condition in which one of two different but normal nucleotide sequences can exist at a particular site in DNA.

The term “restriction enzyme” as used herein refers to an endonuclease, isolated from bacteria, that cleaves DNA at a specific nucleotide sequence.

The term “restriction fragment length polymorphism” or “RFLP” as used herein refers to a method of genetic analysis that examines polymorphisms based on differences in the number of fragments produced by the digestion of DNA with specific endonucleases. In regions of the human genome not coding for proteins there often is wide sequence variety between individuals that can be measured; in effect, the distance (in nucleotides on the chromosome) can be different, usually because of repeated base patterns.

The term “restriction length polymorphism” or “fragment length polymorphism” refers to the existence of allelic forms recognizable by the length of fragments that result when the nucleotide chain is treated by a specific restriction enzyme that cleaves wherever a particular sequence of nucleotides occurs. A change/mutation in this sequence changes cleaving and hence the number of fragments.

The term “restriction site polymorphism” refers to a DNA polymorphism in which the sequence of one form of the polymorphism contains a recognition site for a particular endonuclease, but the sequence of another form lacks such a site.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity”.

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (available on the world wide web (www) at the URL “ncbi.nlm.nih.gov”). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Clayerie and States, Comput. Chem., 17:191-201 (1993)) low-complexity filters may be employed alone or in combination.

The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid.

A “variant” of a gene or nucleic acid sequence is a sequence having at least 65% identity with the referenced gene or nucleic acid sequence, and can include one or more base deletions, additions, or substitutions with respect to the referenced sequence. The differences in the sequences may by the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” of the original sequence.

The term “specifically hybridizes” as used herein refers to the process whereby a nucleic acid distinctively or definitively forms base pairs (bps) with complementary regions of at least one strand of the nucleic acid target sequence that was not originally paired to the nucleic acid. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, at least 85% sequence identity, at least 90% sequence identity, at least 95% sequence identity, or at least 100% sequence identity (i.e., complementary) with each other.

### Method for Assessing a Genetic Risk of Developing Late-Onset Alzheimer's Disease (LOAD) in a Subject

According to one aspect, the described invention provides a method for assessing a genetic risk of developing late-onset Alzheimer's disease in a subject, the method comprising:

(a) isolating a genomic deoxyribonucleic acid (gDNA) from the subject;

(b) amplifying a first genomic region of the genomic deoxyribonucleic acid (gDNA) from step (a) using a first forward primer (5′-TAA GCT TGG CAC GGC TGT CCA AGG A-3′; SEQ ID NO: 1) and a first reverse primer (5′-ACA GAA TTC GCC CCG GCC TGG TAC AC-3′; SEQ ID NO: 2),

wherein the first genomic region comprises genomic deoxyribonucleic acid (gDNA) encoding amino acid positions 112 and 158 of Apolipoprotein E (APOE),

wherein Apolipoprotein E (APOE) isoforms, ε2, ε3, and ε4 contain two single nucleotide polymorphisms (SNPs) at the amino acid positions 112 and 158 of Apolipoprotein E (APOE),

wherein the first forward primer and the first reverse primer flank the two single nucleotide polymorphisms (SNPs), and

wherein the amplification produces a first amplified deoxyribonucleic acid (DNA) with a length of 244 base pair;

(c) amplifying a second genomic region of the genomic deoxyribonucleic acid (gDNA) from step (a) using a second forward primer (5′-GTC TCC AAC TGC TGA CCT C-3′; SEQ ID NO: 3) and a second reverse primer (5′-CTG CCT TTT CAA GCC TCA G-3′; SEQ ID NO: 4),

wherein the second genomic region comprises intron 6 of Translocase of Outer Mitochondrial Membrane 40 homolog gene (TOMM40) containing a polymorphic region of poly-thymidine (poly-T),

wherein the second forward primer and the second reverse primer flank the polymorphic region of the Translocase of Outer Mitochondrial Membrane 40 homolog gene (TOMM40), and

wherein the amplification produces a second amplified deoxyribonucleic acid (DNA) with a length of from about 360 base pair to about 390 base pair;

(d) digesting the first amplified deoxyribonucleic acid (DNA) from step (b) with restriction enzyme HhaI, wherein the restriction enzyme HhaI differentially cleaves the two single nucleotide polymorphisms (SNPs) located at amino acid positions 112 and 158 of Apolipoprotein E (APOE) thereby produces a first restriction fragment length polymorphism (RFLP) comprising:

(i) a 72 base pair fragment,

(ii) an 81 base pair fragment or

(iii) absence of either the 72 base pair fragment or the 81 base pair fragment;

(e) digesting the second amplified deoxyribonucleic acid (DNA) from step (c) with restriction enzyme SmaI, wherein the restriction enzyme SmaI produces a second Restriction Fragment Length Polymorphism (RFLP) comprising

(i) a constant length restriction fragment of about 230 base pair independent of the poly-thymidine (Poly-T) region, and

(ii) a variable-length restriction fragment with a nucleotide length of from about 130 to about 160 base pair;

(f) analyzing haplotypes of the Apolipoprotein E gene (APOE) in the subject based on the first restriction fragment length polymorphism (RFLP) produced by step (d),

wherein presence of the 72 base pair fragment indicates that the subject has an ε4 allele for the Apolipoprotein E gene (APOE),

wherein presence of the 81 base pair fragment indicates that the subject has an ε2 allele for the Apolipoprotein E gene (APOE), and

wherein absence of either the 72 base pair fragment or the 81 base pair fragment indicates that the subject has an ε3 allele for the Apolipoprotein E gene (APOE);

(g) analyzing haplotypes of Translocase of Outer Mitochondrial Membrane 40 homolog gene (TOMM40) in the subject based on the second restriction fragment length polymorphism (RFLP) produced by step (e),

wherein presence of the variable-length restriction fragment of between 15 and 19 thymidine residues indicates that the subject has a short poly-T allele for the Translocase of Outer Mitochondrial Membrane 40 homolog gene (TOMM40),

wherein presence of the variable-length restriction fragment of between 20 and 29 thymidine residues indicates that the subject has a long poly T allele for the Translocase of Outer Mitochondrial Membrane 40 homolog gene (TOMM40),

wherein presence of the variable-length restriction fragment of between 30 and 39 thymidine residues indicates that the subject has a very long poly T allele for the Translocase of Outer Mitochondrial Membrane 40 homolog gene (TOMM40); and

(h) determining the genetic risk for late-onset Alzheimer's disease (LOAD) in the subject based upon the haplotype analysis of the Apolipoprotein E gene (APOE) and the Translocase of Outer Mitochondrial Membrane 40 homolog gene (TOMM40) obtained from step (f) and step (g),

wherein the subject is diagnosed as being in a most at-risk group for late-onset Alzheimer's disease (LOAD) if homozygous ε4 alleles (ε4/ε4) for the Apolipoprotein E gene (APOE) are present in the subject,

wherein the subject is diagnosed as being in an increased risk group for late-onset Alzheimer's disease (LOAD) if heterozygous ε3 and ε4 alleles (ε3/ε4) for the Apolipoprotein E gene (APOE), homozygous ε3 alleles (ε3/ε3) for the Apolipoprotein E gene (APOE), or heterozygous ε2 and ε3 alleles (ε2/ε3) for the Apolipoprotein E gene (APOE) are present together with either the long poly T allele or the very long poly T allele for the Translocase of Outer Mitochondrial Membrane 40 homolog gene (TOMM40) in the subject.

According to one embodiment of the method, the genomic deoxyribonucleic acid (gDNA) is isolated from cerebrospinal fluid (CSF) of the subject.

According to another embodiment, the genomic deoxyribonucleic acid (gDNA) is isolated from peripheral blood of the subject.

According to another embodiment, amplification steps (b) and (c) are carried out by a polymerase chain reaction (PCR).

According to another embodiment, amplification steps (b) and (c) are carried out in a single polymerase chain reaction (PCR).

According to another embodiment, digesting steps (d) and (e) are carried out in a single restriction reaction.

According to another embodiment, digestion step (d) is carried out using an isoschizomer of the restriction enzyme HhaI.

According to another embodiment, digestion step (e) is carried out using an isoschizomer of the restriction enzyme SmaI.

According to another embodiment, in step (h) the subject in the increased risk group for late-onset Alzheimer's disease (LOAD) has heterozygous ε3 and ε4 alleles (ε3/ε4) for the Apolipoprotein E gene (APOE) gene together with either the long poly T allele or the very long poly T allele for the Translocase of Outer Mitochondrial Membrane 40 homolog gene (TOMM40).

According to another embodiment, in step (h) the subject in the increased risk group for late-onset Alzheimer's disease (LOAD) contains homozygous ε3 alleles (ε3/ε3) for the Apolipoprotein E gene (APOE) together with either the long poly T allele or the very long poly T allele for the Translocase of Outer Mitochondrial Membrane 40 homolog gene (TOMM40).

According to another embodiment, in step (h) the subject in the increased risk group for late-onset Alzheimer's disease (LOAD) contains heterozygous ε2 and ε3 alleles (ε2/ε3) for the Apolipoprotein E gene (APOE) together with either the long poly T allele or the very long poly T allele for the Translocase of Outer Mitochondrial Membrane 40 homolog gene (TOMM40).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Extraction of Genomic DNA

Human hippocampal brain tissue from Alzheimer's disease (AD) and non-AD (control) subjects was collected at the specified post-mortem interval (PMI) (Table 1; hours) and stored at −80° C. for future processing. Genomic DNA (gDNA) was extracted and purified from the brain tissue using the Wizard SV Genomic DNA Purification System, according to manufacturer's suggested guidelines. Briefly, 20 mg of tissue was enzymatically digested overnight at 55° C. in a solution containing proteinase K and RNase A. The following day, samples were centrifuged at 2000×g and supernatants were transferred and bound to spin columns provided within the kit. gDNA was eluted in 65° C. nuclease-free H₂O and DNA concentration measured with a spectrophotometer. gDNA was run on a 1% agarose gel to confirm the presence of high molecular weight (>10 kb) product and absence of degradation (data not shown).

### PCR Amplification and Restriction Digest

200 ng of genomic DNA (gDNA) was used as template for PCR amplification of APOE (Accession: NG_{—}007084.2) (+2839/+3066) and TOMM40 (Accession: NC_{—}000019.9) (+8489/+8874) with Platinum Taq DNA Polymerase (Invitrogen, Carlsbad, Calif.). The primer sequences were: APOE (F) 5′-taa get tgg cac ggc tgt cca agg a-3′ (SEQ ID NO: 1) and (R) 5′-aca gaa ttc gcc ccg gcc tgg tac ac-3′ (SEQ ID NO: 2); TOMM40 (F) 5′-gtc tcc aac tgc tga cct c-3′ (SEQ ID NO: 3): and (R) 5′-ctg cct ttt caa gcc tca g-3′ (SEQ ID NO: 4). The cycling profile for APOE and TOMM40 (45 cycles) was: 95° C. for 45 sec, 58° C. for 45 sec and 72° C. for 60 sec with a final extension at 72° C. for 10 min. The resulting PCR products were analyzed by gel electrophoresis on 1% agarose gels containing ethidium bromide, and imaged using a Biorad (Hercules, Calif.) Chemidoc XRS System and Quantity One software (Biorad).

To generate the APOE and TOMM40 restriction fragment length polymorphisms (RFLPs), respective PCR products were purified using the Wizard SV Gel and PCR Clean-Up System according to manufacturers suggested guidelines. Briefly, membrane binding solution was added to the fresh PCR product and the total volume bound to the spin column provided within the kit. PCR product was eluted in nuclease-free H₂O and DNA concentration measured with a spectrophotometer. The product was also run on a 1% agarose gel to confirm the presence of a single DNA band (data not shown). 1 μg of purified product was then digested with either HhaI (for APOE) or SmaI (for TOMM40) Fast Digest restriction enzyme (Fermentas, Glen Burnie, Md.) for 1 h at 37° C. Digested products were analyzed on 4% agarose gels and imaged.

### DNA Sequencing

Confirmatory identification of the TOMM40 sequence was determined by DNA sequencing of the purified PCR product. All sequencing reactions were performed by Genewiz, Inc. (South Plainfield, N.J.) using the following primer: 5′-tac agg cca caa atg tga-3′ (SEQ ID NO: 5). Sequence alignment to Accession: NC_{—}000019.9 was carried out using the NCBI pairwise alignment tool (bl2seq) (available on world wide web at the URL “blast.ncbi.nlm.nih.gov/Blast.cgi”).

### Results

### APOE Genotyping by PCR and RFLP

APOE genotypes of samples from both AD and control patients (Table 1) were enumerated using a previously established method based on gene amplification and cleavage with the restriction enzyme, HhaI, to generate RFLPs (Hixson and Powers, J Lipid Res, 1991; 32: 1529-1535).

TABLE 1
Sample Information for AD and Controls used in APOE and TOMM40
Haplotyping

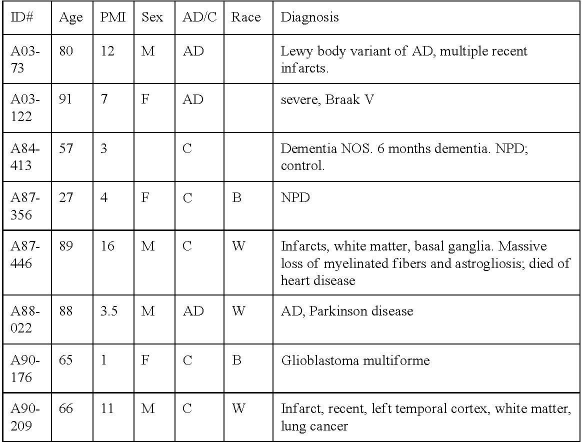

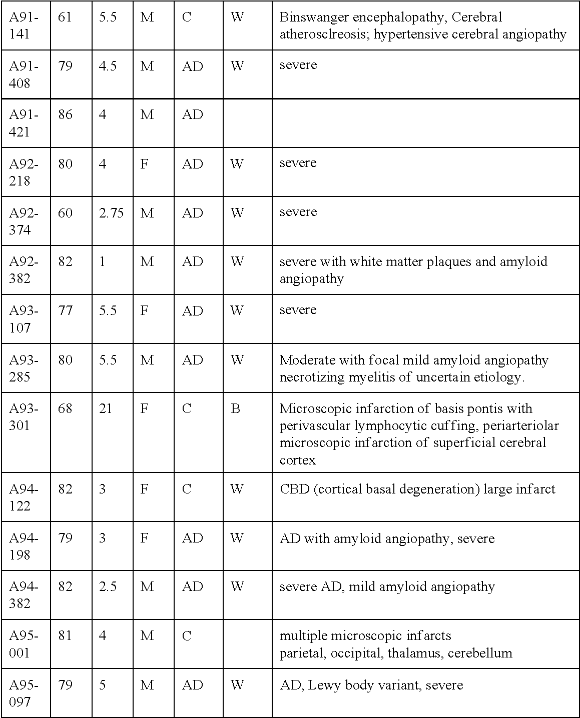

Brain tissue was collected at the specified post-mortem interval (PMI; hours) and stored (−80° C.) for future analysis. Genomic DNA extracted from hippocampal tissue was used for all genotyping studies. Shaded sections in Table 1 were used as a representative sample population for the illustrated genotyping results.

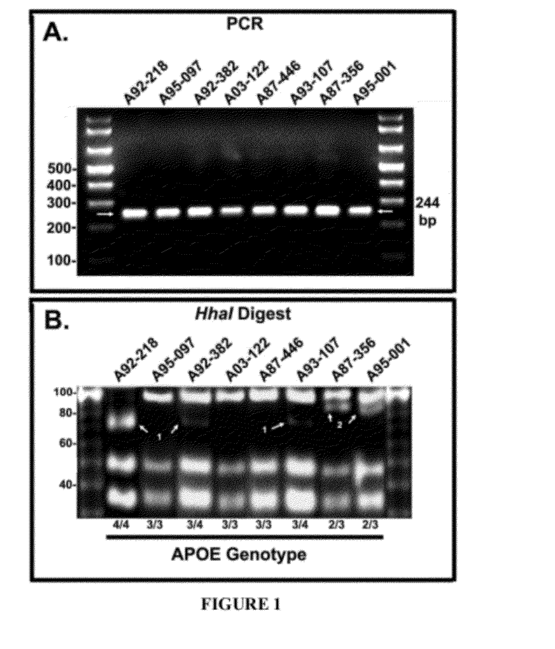

The three APOE isoforms, ε2, ε3 and ε4, differ by a single nucleotide at amino acid positions 112 and 158, thus primers flanking these single nucleotide polymorphisms (SNPs) were first utilized to amplify the region by PCR (FIG. 1A). Depending on the individual APOE isotype, HhaI cleaves between 4 and 6 sites within this 244 bp region, and differentially cleaves the two SNP sites to generate a unique pattern of Restriction Fragment Length Polymorphisms (RFLPs) that can be analyzed on a 4% agarose gel for genotyping (FIG. 1B). The distinguishing bands are a 72 bp fragment (arrow 1) unique to the ε4 isotype and an 81 bp fragment (arrow 2) unique to the ε2 isotype. Neither of these fragments is generated by cleaving the ε3 isotype. Table 2 lists the complete APOE genotypes for the 22 AD and control samples tested.

TABLE 2
APOE Genotypes Determined by RFLP

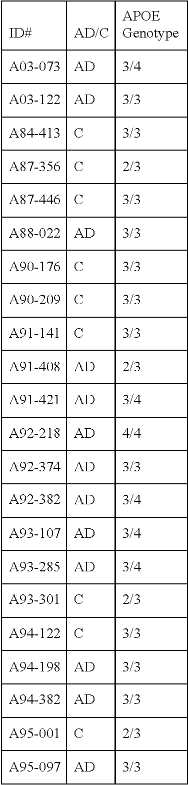

The PCR-Amplified gDNA corresponding to the APOE polymorphic region was digested with HhaI and separated on a 4% agarose gel. Restriction fragment sizes were used to assess APOE genotypes. Shaded sections in Table 2 were used as a representative sample population as illustrated in FIG. 1.

### Isotyping the TOMM40 Poly-T Variable Region

APOE and TOMM40 are adjacent to each other on chromosome 19, separated by a mere about 2 kb, and have been shown to be in genetic linkage disequilibrium (Martin et al., Am J Hum Genet, 67: 383-394, 2000; Takei et al., Genomics, 93: 441-448, 2009; Yu et al., Genomics, 2007; 89: 655-65). The TOMM40 poly-T polymorphism is distinguished by short (<20 T residues), long (20-30 T residues) and very long (>30 T residues) isoforms which are phylogenetically linked to APOE (Roses et al., Pharmacogenomics J, 10: 375-84, 2010). The ε4 allele appears to be inherited together with the long variant, while the ε3 allele associates with either the short or very long TOMM40 variants. ε3/ε4 individuals possessing two long poly-T alleles develop LOAD on average 7 years earlier than individuals possessing one long and one short allele (Roses et al., Pharmacogenomics J, 10: 375-84, 2010).

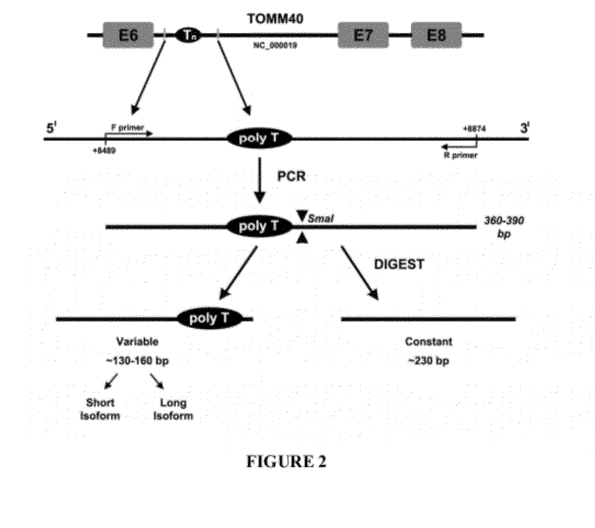

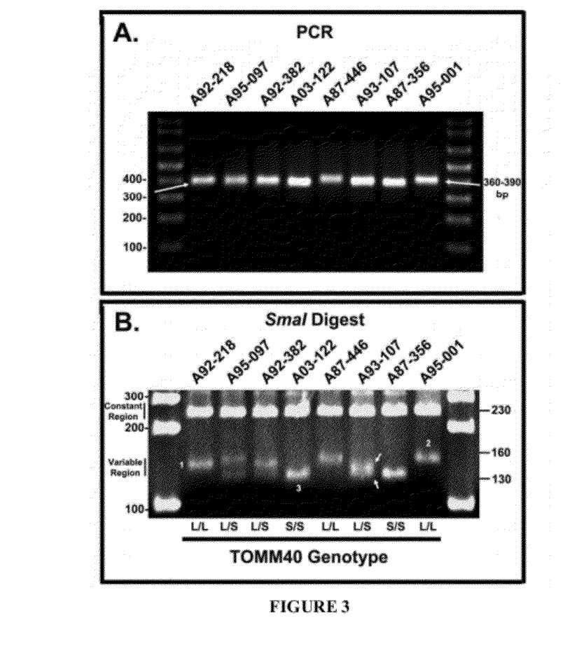

According to the described invention, a method similar to that used for APOE genotyping (FIG. 1) was sought to distinguish between the TOMM40 polymorphic variants. The impetus behind this approach was that APOE and TOMM40 haplotyping of the same sample could occur within one benchtop workflow. As illustrated in FIG. 2, primers were designed to amplify the regions spanning the TOMM40 polymorphic region within intron 6. The resulting PCR amplicon varied in length between 360-390 bp depending on the number of T residues within the polymorphic region (FIG. 3A). Just 3′ of the poly-T region is a cleavage site for the restriction enzyme, SmaI (FIG. 2), which was utilized to digest the TOMM40 amplicon into two fragments: (1) a poly-T-excluding fragment with a constant size of 230 bp; and (2) a poly-T-including fragment with a variable size between 130-160 bp depending on the allelic variant. The size differences between 130-160 bp fragments were resolved using a 4% agarose gel, which allows genotyping of the TOMM40 poly-T region (FIG. 3B). There were clear size distinctions between the long (labels 1, 2) and short (label 3) poly-T variants with this approach, which allows determination of whether samples were homo- or heterozygous for each variant. The fragments were identified as either short (S) or long (L) for the purpose of this study, however there does appear to be a difference in size among the long variants found in ε4 (long; “1”) versus ε3 or ε2 (very long; “2”) isotypes. Table 3 lists the complete APOE and TOMM40 haplotypes for the 22 AD and control samples tested.

TABLE 3
TOMM40 Genotypes Determined by RFLP

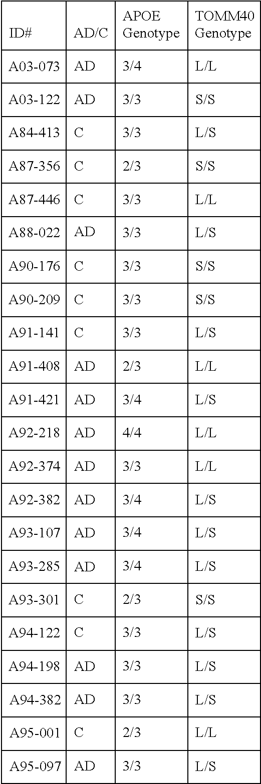

PCR Amplified gDNA corresponding to the intronic region between exons 6 and 7 of the TOMM40 gene containing the poly-T hypervariable region (FIG. 2) was digested with SmaI and separated on a 4% agarose gel. Restriction fragment sizes were used to assess TOMM40 genotypes. Shaded sections were used as a representative sample population as illustrated in FIG. 3. (L=long variant; S=short variant)

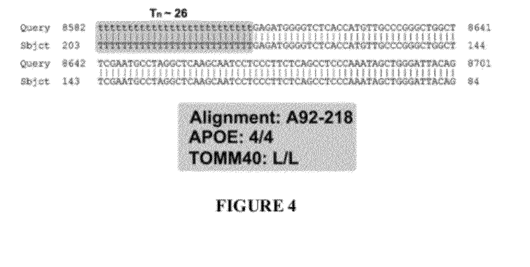

To validate the identity of the TOMM40 amplicon and to quantify the number of T residues in a given sample, the PCR amplicon was sequenced (FIG. 4). Direct sequencing of a PCR product from genomic DNA (gDNA) is difficult when there is heterozygosity, since both alleles may be primed and the nucleotide reading frame lost once the variable segment is encountered. In addition, sequencing of a region consisting of many sequential T residues can be troublesome. Therefore, the PCR amplicon was sequenced from a long variant homozygote (A92-218), and the result was aligned with a known TOMM40 long variant (GenBank Accession # NC_{—}000019.9) using the NCBI pairwise alignment tool (bl2seq). The results confirmed both the identity of the PCR product and the restriction-based assessment of this sample as a long poly-T variant (26 residues). However, the reading frame was lost after the last T residue, suggesting a slight variation in the number of T residues among the individual isotypes. These findings indicate that the PCR- and restriction-based method is most accurate for qualitative (short versus long) assessment of TOMM40 variant length.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for assessing a genetic risk of developing late-onset Alzheimer's disease (LOAD) in a subject, the method comprising:
(a) isolating a genomic deoxyribonucleic acid (gDNA) from the subject;
(b) amplifying a first genomic region of the genomic deoxyribonucleic acid (gDNA) from step (a) using a first forward primer (5′-TAA GCT TGG CAC GGC TGT CCA AGG A-3′; SEQ ID NO: 1) and a first reverse primer (5′-ACA GAA TTC GCC CCG GCC TGG TAC AC-3′; SEQ ID NO: 2),
wherein the first genomic region comprises genomic deoxyribonucleic acid (gDNA) encoding amino acid positions 112 and 158 of Apolipoprotein E (APOE),
wherein Apolipoprotein E (APOE) isoforms ε2, ε3, and ε4 contain two single nucleotide polymorphisms (SNPs) at the amino acid positions 112 and 158 of Apolipoprotein E (APOE),
wherein the first forward primer and the first reverse primer flank the two single nucleotide polymorphisms (SNPs), and
wherein the amplification produces a first amplified deoxyribonucleic acid (DNA) with a length of 244 base pair;
(c) amplifying a second genomic region of the genomic deoxyribonucleic acid (gDNA) from step (a) using a second forward primer (5′-GTC TCC AAC TGC TGA CCT C-3′; SEQ ID NO: 3) and a second reverse primer (5′-CTG CCT TTT CAA GCC TCA G-3′; SEQ ID NO: 4),
wherein the second genomic region comprises intron 6 of Translocase of Outer Mitochondrial Membrane 40 homolog gene (TOMM40) containing a polymorphic region of poly-thymidine (poly-T),
wherein the second forward primer and the second reverse primer flank the polymorphic region of the Translocase of Outer Mitochondrial Membrane 40 homolog gene (TOMM40), and
wherein the amplification produces a second amplified deoxyribonucleic acid (DNA) with a length of from about 360 base pair to about 390 base pair;
(d) digesting the first amplified deoxyribonucleic acid (DNA) from step (b) with restriction enzyme HhaI, wherein the restriction enzyme HhaI differentially cleaves the two single nucleotide polymorphisms (SNPs) located at amino acid positions 112 and 158 of Apolipoprotein E (APOE) thereby produces a first Restriction Fragment Length Polymorphism (RFLP) comprising
(i) a 72 base pair fragment,
(ii) an 81 base pair fragment or
(iii) absence of either the 72 base pair fragment or the 81 base pair fragment;
(e) digesting the second amplified deoxyribonucleic acid (DNA) from step (c) with restriction enzyme SmaI, wherein the restriction enzyme SmaI produces a second Restriction Fragment Length Polymorphism (RFLP) comprising
(i) a constant length restriction fragment of about 230 base pair independent of the poly-thymidine (Poly-T) region, and
(ii) a variable-length restriction fragment with a nucleotide length of from about 130 to about 160 base pair;
(f) analyzing haplotypes of the Apolipoprotein E gene (APOE) in the subject based on the first restriction fragment length polymorphism (RFLP) produced by step (d),
wherein presence of the 72 base pair fragment indicates that the subject has an ε4 allele for the Apolipoprotein E gene (APOE),
wherein presence the 81 base pair fragment indicates that the subject has an ε2 allele for the Apolipoprotein E gene (APOE), and
wherein absence of either the 72 base pair fragment or the 81 base pair fragment indicates that the subject has an ε3 allele for the Apolipoprotein E gene (APOE);
(g) analyzing haplotypes of Translocase of Outer Mitochondrial Membrane 40 homolog gene (TOMM40) in the subject based on the second restriction fragment length polymorphism (RFLP) produced by step (e),
wherein presence of the variable-length restriction fragment of between 15 and 19 thymidine residues indicates that the subject has a short poly-T allele for the Translocase of Outer Mitochondrial Membrane 40 homolog gene (TOMM40),
wherein presence of the variable-length restriction fragment of between 20 and 29 thymidine residues indicates that the subject has a long poly-T allele for the Translocase of Outer Mitochondrial Membrane 40 homolog gene (TOMM40),
wherein presence of the variable-length restriction fragment of between 30 and 39 thymidine residues indicates that the subject has a very long poly-T allele for the Translocase of Outer Mitochondrial Membrane 40 homolog gene (TOMM40); and
(h) determining the subject's genetic risk for late-onset Alzheimer's disease (LOAD) based upon the haplotype analysis of the Apolipoprotein E gene (APOE) and the Translocase of Outer Mitochondrial Membrane 40 homolog gene (TOMM40) obtained from step (f) and step (g),
wherein the subject is diagnosed as being in a most at-risk group for late-onset Alzheimer's disease (LOAD) if homozygous ε4 alleles (ε4/ε4) for the Apolipoprotein E gene (APOE) are present in the subject,
wherein the subject is diagnosed as being in an increased risk group for late-onset Alzheimer's disease (LOAD) if heterozygous ε3 and ε4 alleles (ε3/ε4) for the Apolipoprotein E gene (APOE), homozygous ε3 alleles (ε3/ε3) for the Apolipoprotein E gene (APOE), or heterozygous ε2 and ε3 alleles (ε2/ε3) for the Apolipoprotein E gene (APOE) are present together with either the long poly-T allele or the very long poly-T allele for the Translocase of Outer Mitochondrial Membrane 40 homolog gene (TOMM40) in the subject.

2. The method according to claim 1, wherein the genomic deoxyribonucleic acid (gDNA) is isolated from cerebrospinal fluid (CSF) of the subject.

3. The method according to claim 1, wherein the genomic deoxyribonucleic acid (gDNA) is isolated from peripheral blood of the subject.

4. The method according to claim 1, wherein amplification steps (b) and (c) are carried out by a polymerase chain reaction (PCR).

5. The method according to claim 1, wherein amplification steps (b) and (c) are carried out in a single polymerase chain reaction (PCR).

6. The method according to claim 1, wherein digesting steps (d) and (e) are carried out in a single restriction reaction.

7. The method according to claim 1, wherein digestion step (d) is carried out using an isoschizomer of the restriction enzyme HhaI.

8. The method according to claim 1, wherein digestion step (e) is carried out using an isoschizomer of the restriction enzyme SmaI.

9. The method according to claim 1, wherein in step (h) the subject in the increased risk group for late-onset Alzheimer's disease (LOAD) has heterozygous ε3 and ε4 alleles (ε3/ε4) for the Apolipoprotein E gene (APOE) gene together with either the long poly-T allele or the very long poly-T allele for the Translocase of Outer Mitochondrial Membrane 40 homolog gene (TOMM40).

10. The method according to claim 1, wherein in step (h) the subject in the increased risk group for late-onset Alzheimer's disease (LOAD) contains homozygous ε3 alleles (ε3/ε3) for the Apolipoprotein E gene (APOE) together with either the long poly-T allele or the very long poly-T allele for the Translocase of Outer Mitochondrial Membrane 40 homolog gene (TOMM40).

11. The method according to claim 1, wherein in step (h) the subject in the increased risk group for late-onset Alzheimer's disease (LOAD) contains heterozygous ε2 and ε3 alleles (ε2/ε3) for the Apolipoprotein E gene (APOE) together with either the long poly-T allele or the very long poly-T allele for the Translocase of Outer Mitochondrial Membrane 40 homolog gene (TOMM40).
